(19) United States
(12) Patent Application Publication
(10) Pub. No.: US 2023/0075950 A1
(43) Pub. Date: Mar. 9, 2023

(54) SYSTEM FOR PROVIDING POWERLINE COMMUNICATION OVER FLEXIBLE MESH FOR CIRCUIT DESIGN USED IN BIOMETRIC MONITORING

(71) Applicant: Cipher Skin, Denver, CO (US)

(72) Inventor: Phillip Bogdanovich, Denver, CO (US)

(73) Assignee: Cipher Skin, Denver, CO (US)

(21) Appl. No.: 17/759,547

(22) PCT Filed: Jan. 28, 2021
(86) PCT No.: PCT/US2021/015543

§ 371 Date: Jul. 27, 2022

Related U.S. Application Data
(60) Provisional application No. 62/967,105, filed on Jan. 29, 2020.

(51) Int. Cl. H04B 3/54

(52) U.S. Cl. CPC H04B 3/548; H04B 3/546; H04B 3/542; H04B 2203/5458

(57) ABSTRACT

A system for providing powerline communication over flexible mesh for circuit design used in biometric monitoring is disclosed. In particular, the system provides for the transmission of power and data over the same circuit infrastructure, and integrates the ability to transfer data over an infrastructure that also transmits power. Notably, the system facilitates the integration of data over power in flexible systems, such as systems incorporating flexible mesh sensors and flexible geometric systems. The dual use of power infrastructure reduces weight and increases flexibility and potential sensor density. The systems use of data over power significantly reduces or eliminates the needs for a standalone data bus, thereby reducing the design complexity of the circuit.

### CROSS REFERENCE TO RELATED APPLICATIONS

This application is a U.S. National Stage entry under 35 U.S.C. § 371 based on International Application No. PCT/US2021/015543, filed on Jan. 28, 2021, which claims priority to U.S. Provisional Patent Application No. 62/967,105, filed on Jan. 29, 2020. These applications are hereby incorporated herein in their entirety by reference.

### FIELD OF THE INVENTION

The present application relates to powerline communication technologies, sensor array technologies, electrical systems, flexible mesh technologies, monitoring technologies, and computing technologies, and more particularly, to a system for providing powerline communication over flexible mesh for circuit design used in monitoring, such as, but not limited to, biometric monitoring.

### BACKGROUND

In today's society, Powerline Communication (PLC) and similar methods of data over power have been utilized in alternating current (AC) systems. For example, PLC has been utilized in AC systems including, home automation systems, and, in earlier instances, military applications. In direct current (DC) systems, the comparable applications are significantly less documented. For example, BMW, Ford, and others have implemented PLC over DC networks in automotive electrical systems for the purposes of monitoring vehicle components as well as creating in car internet connections. Commonalities between all existing implementations of PLC are that the circuit is specific in its design and use, and the architecture is purpose-built. Notably, however, nothing exists that provides data over power in the manner as described in the present disclosure. Furthermore, nothing exists where data over power is used as a method of transferring data collected relevant to changes in the circuit itself.

PLC typically functions such that there are master nodes or hubs where each hub contains a transmitter (TX) and receiver (RX) as well as a decoder circuit that allows for information to be collected at a node, packaged, and transmitted during power transmission over an electrical circuit/wires. Hubs are typically large, analog devices that consume significant amounts of power, take up space, and are comparably heavy relative to other circuit components. PLC in this format is also limited in its network size due to the limitations of the TX/RX module arrangement. Additionally, garment sensor arrays and circuits are bulky and limited in their size and application. The requirements for power and data lead to a circuit that is large in size, difficult to scale and specific in their application to account for power consumption and data transfer needs.

Based on the foregoing, current technologies and processes may be modified and improved so as to provide enhanced functionality and features for users and systems to effectively provide PLC over DC and other systems. Such enhancements and improvements may provide for improved user satisfaction, increased reliability, increased efficiencies, increased access to meaningful data, increased communications capabilities, substantially reduced costs for businesses and individuals, and increased ease-of-use for users.

### SUMMARY

A system and methods for providing powerline communication over flexible mesh for circuit design used in monitoring are disclosed. In particular, the system and methods enable the transmission of data and power in a scalable flexible mesh infrastructure using direct current as a primary power source. Additionally, the system and methods utilize data over power as a method for transferring data that is collected via the flexible mesh infrastructure that is relevant to changes in the circuitry of the flexible mesh infrastructure. By enabling transmission of data and power using direct current, the system and methods reduce weight of the infrastructure needed for transmitted data and power and allow for the digital mapping of freedom and constraint topology. To that end, the system and methods enable each sensor of the flexible mesh infrastructure to be capable of transmission and receipt of power and data. By having such functionality, the system and methods eliminate the need for hubs to manage the compression, transmission, and receipt of data over an existing infrastructure, which reduces the design complexity of the circuitry of the infrastructure. Thus, the system and methods provide for a solution for monitoring that is light, flexible, and scalable, while enabling the transmission of power and data over the same circuit infrastructure.

Notably, the system and methods may be adapted to accommodate various methods of data transfer using the flexible mesh infrastructure. For example, the system and methods may utilize powerline communication, digital data burst over electrical infrastructure, and coded, multiplexed analog to digital/digital to analog transmission. In certain embodiments, the system and methods may be configured to integrate data over power in flexible geometric systems. The dual use of power infrastructure reduces weight and increases flexibility and potential sensor density as well. In certain embodiments, the system and methods account for changes in electrical properties associated with the distortion of conductive elastomers of the flexible mesh infrastructure and work well in conductive elastomer environments. Furthermore, the system and methods may utilize the flexible mesh infrastructure to conduct any type of monitoring including, but not limited to, biometric monitoring of users, any type of monitoring, or a combination thereof. In certain embodiments, the flexible mesh infrastructure may be configured to monitor any object upon which the flexible mesh infrastructure is disposed on. For example, the flexible mesh circuits and/or infrastructure may be disposed on and may be utilized to monitor pipes, buildings, bodies, animals, equipment, vehicles, plants, trees, any type of terrain, any type of body of water, any type of object, or any combination thereof.

To that end, in one embodiment according to the present disclosure, a system for providing powerline communication over flexible mesh for circuit design used in monitoring is disclosed. The system may include a flexible mesh circuit infrastructure that includes a plurality of sensors arranged within and/or on the flexible mesh circuit infrastructure. In certain embodiments, each sensor of the plurality of sensors may be configured to transmit and receive data and power in the flexible mesh circuit infrastructure using powerline communication and by using direct current as a primary power source.

In another embodiment, a method for providing powerline communication over flexible mesh for circuit design used in monitoring is disclosed. The method may include providing a flexible mesh circuit infrastructure. Additionally, the method may include arranging a plurality of sensors within the flexible mesh circuit infrastructure. Furthermore, the method may include facilitating transmission and receipt of data and power from each sensor of the plurality of sensors in the flexible mesh circuit infrastructure by utilizing powerline communication and by utilizing direct current as a primary power source.

In yet another embodiment, a flexible mesh circuit infrastructure is disclosed. The flexible mesh circuit infrastructure may include a plurality of sensors, wherein each sensor of the plurality of sensors may be configured to transmit and receive data and power using powerline communication and by using direct current as a primary power source.

These and other features of the systems and methods for providing powerline communication over flexible mesh for circuit design used in monitoring are described in the following detailed description, drawings, and appended claims.

### BRIEF DESCRIPTION OF THE DRAWINGS

FIG. 1 is a schematic diagram of a system for providing powerline communication over flexible mesh for circuit design used in monitoring according to an embodiment of the present disclosure.

FIG. 2 is a flow diagram illustrating a sample method for providing powerline communication over flexible mesh for circuit design used in monitoring according to an embodiment of the present disclosure.

FIG. 3 is a schematic diagram of a machine in the form of a computer system within which a set of instructions, when executed, may cause the machine to perform any one or more of the methodologies or operations of the systems and methods for providing powerline communication over flexible mesh for circuit design used in monitoring.

### DETAILED DESCRIPTION OF THE INVENTION

A system 100 and methods for providing powerline communication over flexible mesh for circuit design used in monitoring are disclosed. In particular, the system 100 and methods enable the transmission of data and power in a scalable flexible mesh infrastructure using direct current as a primary power source. Additionally, the system 100 and methods utilize data over power as a method for transferring data that is collected via the flexible mesh infrastructure that is relevant to changes in the circuitry of the flexible mesh infrastructure. By enabling transmission of data and power using direct current over the same infrastructure, the system and methods reduce weight of the infrastructure needed for transmitted data and power and allow for the digital mapping of freedom and constraint topology. The system 100 and methods enable each sensor of the flexible mesh infrastructure to be capable of transmission and receipt of power and data. By having such functionality, the system 100 and methods eliminate the need for hubs to manage the compression, transmission, and receipt of data over an existing infrastructure, which reduces the design complexity of the circuitry of the infrastructure itself. Thus, the system 100 and methods provide for a solution for monitoring that is light, flexible, and scalable, while enabling the transmission of power and data over the same circuit infrastructure.

In certain embodiments, the system 100 and methods may be adapted to accommodate various methods of data transfer using the flexible mesh infrastructure. For example, the system 100 and methods may utilize powerline communication, digital data burst over electrical infrastructure, and coded, multiplexed analog to digital/digital to analog transmission. In certain embodiments, the system 100 and methods may be configured to integrate data over power in flexible geometric systems. The dual use of power infrastructure reduces weight and increases flexibility and potential sensor density as well. In certain embodiments, the system 100 and methods account for changes in electrical properties associated with the distortion of conductive elastomers of the flexible mesh infrastructure and work well in conductive elastomer environments. Furthermore, the system 100 and methods may utilize the flexible mesh infrastructure to conduct any type of monitoring including, but not limited to, biometric monitoring of users, any type of monitoring, or a combination thereof. In certain embodiments, the flexible mesh infrastructure may be configured to monitor any object upon which the flexible mesh infrastructure is disposed on.

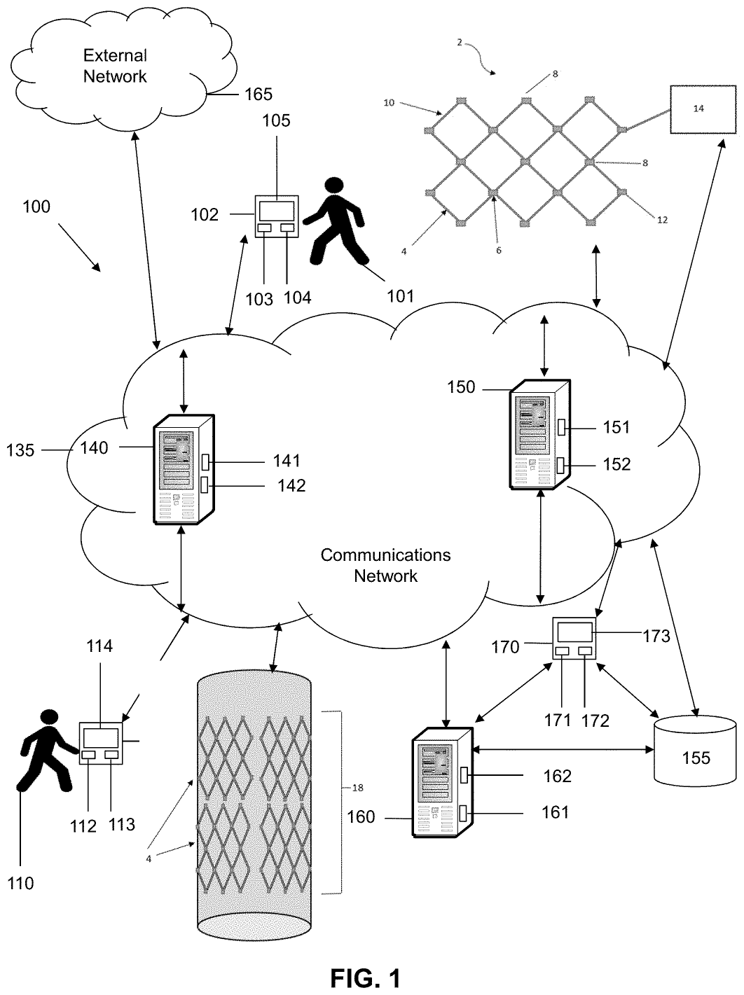

As shown in FIGS. 1-3, a system 100 and method for providing powerline communication over flexible mesh for circuit design used in monitoring is disclosed. The system 100 may be configured to support, but is not limited to supporting, monitoring applications and services, sensor-based applications and services, wearable device applications and services, health monitoring applications and services, communication applications and services, alert applications and services, data and content services, data aggregation applications and services, big data technologies, health analysis technologies, data synthesis applications and services, data analysis applications and services, computing applications and services, cloud computing services, internet services, satellite services, telephone services, software as a service (SaaS) applications, mobile applications and services, and any other computing applications and services. The system may include a first user 101, who may utilize a first user device 102 to access data, content, and applications, or to perform a variety of other tasks and functions. As an example, the first user 101 may utilize first user device 102 to access an application (e.g. a browser or a mobile application) executing on the first user device 102 that may be utilized to access web pages, data, and content associated with the system 100. In certain embodiments, the first user 101 may be a user that is a worker at an industrial plant, oil refinery, ship, factory, and/or any other location that may seek to be monitored, such as by utilizing flexible mesh as described in the present disclosure.

The first user device 102 utilized by the first user 101 may include a memory 103 that includes instructions, and a processor 104 that executes the instructions from the memory 103 to perform the various operations that are performed by the first user device 102. In certain embodiments, the processor 104 may be hardware, software, or a combination thereof. The first user device 102 may also include an interface 105 (e.g. screen, monitor, graphical user interface, audio device interface, etc.) that may enable the first user 101 to interact with various applications executing on the first user device 102, to interact with various applications executing within the system 100, and to interact with the system 100 itself. In certain embodiments, the first user device 102 may be a computer, a laptop, a tablet device, a phablet, a server, a mobile device, a smartphone, a smart watch, and/or any other type of computing device. Illustratively, the first user device 102 is shown as a mobile device in FIG. 1. The first user device 102 may also include a global positioning system (GPS), which may include a GPS receiver and any other necessary components for enabling GPS functionality, accelerometers, gyroscopes, sensors, and any other componentry suitable for a mobile device. In certain embodiments, the first user device 102 may be configured to include any number of sensors, such as, but not limited to, temperature sensors, pressure sensors, motion sensors, light sensors, oxygen sensors, heart rate sensors, touch sensors, proximity sensors, gas sensors, acoustic sensors, chemical sensors, acceleration sensors, humidity sensors, moisture sensors, presence sensors, force sensors, any type of sensors, or a combination thereof.

In addition to the first user 101, the system 100 may include a second user 110, who may utilize a second user device 111 to access data, content, and applications, or to perform a variety of other tasks and functions. As with the first user 101, the second user 110 may be a user that is a worker at an industrial plant, oil refinery, ship, factory, and/or any other location as well. However, in certain embodiments, the second user 110 may be a supervisor of the first user 101, a physician, a first responder, an emergency personnel, a nurse, any type of health professional, any type of safety personnel, or any combination thereof. Much like the first user 101, the second user 110 may utilize second user device 111 to access an application (e.g. a browser or a mobile application) executing on the second user device 111 that may be utilized to access web pages, data, and content associated with the system 100. The second user device 111 may include a memory 112 that includes instructions, and a processor 113 that executes the instructions from the memory 112 to perform the various operations that are performed by the second user device 111. In certain embodiments, the processor 113 may be hardware, software, or a combination thereof. The second user device 111 may also include an interface 114 (e.g. a screen, a monitor, a graphical user interface, etc.) that may enable the second user 110 to interact with various applications executing on the second user device 111, to interact with various applications executing in the system 100, and to interact with the system 100. In certain embodiments, the second user device 111 may be a computer, a laptop, a tablet device, a phablet, a server, a mobile device, a smartphone, a smart watch, and/or any other type of computing device. Illustratively, the second user device 111 may be a computing device in FIG. 1. The second user device 111 may also include any of the componentry described for first user device 102.

In certain embodiments, the first user device 102 and the second user device 111 may have any number of software applications and/or application services stored and/or accessible thereon. For example, the first and second user devices 102, 111 may include applications for facilitating the transmission of data and/or power, determining and analyzing health conditions, applications for determining and analyzing the physiological status of a user, applications for generating alerts, applications for analyzing and interpreting sensor data, artificial intelligence applications, machine learning applications, big data applications, applications for analyzing data, applications for integrating data, cloud-based applications, search engine applications, natural language processing applications, database applications, algorithmic applications, phone-based applications, product-ordering applications, business applications, e-commerce applications, media streaming applications, content-based applications, database applications, gaming applications, internet-based applications, browser applications, mobile applications, service-based applications, productivity applications, video applications, music applications, social media applications, presentation applications, any other type of applications, any types of application services, or a combination thereof. In certain embodiments, the software applications and services may include one or more graphical user interfaces so as to enable the first and second users 101, 110 to readily interact with the software applications.

The software applications and services may also be utilized by the first and second users 101, 110 to interact with any device in the system 100, any network in the system 100, or any combination thereof. For example, the software applications executing on the first and second user devices 102, 111 may be applications for receiving data, applications for storing data, applications for determining health conditions, applications for determining how to respond to a health condition, applications for determining a physiological status of a user, applications for determining how to respond to an environmental condition (e.g. an environmental condition that may affect the first user 101), applications for receiving demographic and preference information, applications for transforming data, applications for executing mathematical algorithms, applications for generating and transmitting electronic messages, applications for generating and transmitting various types of content, any other type of applications, or a combination thereof. In certain embodiments, the first and second user devices 102, 111 may include associated telephone numbers, internet protocol addresses, device identities, or any other identifiers to uniquely identify the first and second user devices 102, 111 and/or the first and second users 101, 110. In certain embodiments, location information corresponding to the first and second user devices 102, 111 may be obtained based on the internet protocol addresses, by receiving a signal from the first and second user devices 102, 111, or based on profile information corresponding to the first and second user devices 102, 111.

The system 100 may also include a communications network 135. The communications network 135 of the system 100 may be configured to link each of the devices in the system 100 to one another. For example, the communications network 135 may be utilized by the first user device 102 to connect with other devices within or outside communications network 135. Additionally, the communications network 135 may be configured to transmit, generate, and receive any information and data traversing the system 100. In certain embodiments, the communications network 135 may include any number of servers, databases, or other componentry, and may be controlled by a service provider. The communications network 135 may also include and be connected to a cloud-computing network, a phone network, a wireless network, an Ethernet network, a satellite network, a broadband network, a cellular network, a private network, a cable network, the Internet, an internet protocol network, a content distribution network, any network, or any combination thereof. Illustratively, server 140 and server 150 are shown as being included within communications network 135.

Notably, the functionality of the system 100 may be supported and executed by using any combination of the servers 140, 150, and 160. The servers 140, and 150 may reside in communications network 135, however, in certain embodiments, the servers 140, 150 may reside outside communications network 135. The servers 140, and 150 may be utilized to perform the various operations and functions provided by the system 100, such as those requested by applications executing on the first and second user devices 102, 111. In certain embodiments, the server 140 may include a memory 141 that includes instructions, and a processor 142 that executes the instructions from the memory 141 to perform various operations that are performed by the server 140. The processor 142 may be hardware, software, or a combination thereof. Similarly, the server 150 may include a memory 151 that includes instructions, and a processor 152 that executes the instructions from the memory 151 to perform the various operations that are performed by the server 150. In certain embodiments, the servers 140, 150, and 160 may be network servers, routers, gateways, switches, media distribution hubs, signal transfer points, service control points, service switching points, firewalls, routers, edge devices, nodes, computers, mobile devices, or any other suitable computing device, or any combination thereof. In certain embodiments, the servers 140, 150 may be communicatively linked to the communications network 135, any network, any device in the system 100, or any combination thereof.

The database 155 of the system 100 may be utilized to store and relay information that traverses the system 100, cache information and/or content that traverses the system 100, store data about each of the devices in the system 100, and perform any other typical functions of a database. In certain embodiments, the database 155 may store the output from any operation performed by the system 100, operations performed and/or outputted by the sensors of the system 100, operations performed and/or outputted by any component, program, process, device, network of the system 100, or any combination thereof. For example, the database 155 may store data from data sources, such as, but not limited to, sensors, which may measure sensor data associated with the first user 101 and/or an environment that the first user 101 is located in. The database 155 may also store information identifying each sensor of the flexible mesh circuit infrastructure and each sensor's functionality and operational status. In certain embodiments, the database 155 may be connected to or reside within the communications network 135, any other network, or a combination thereof. The database 155 may also store communications transmitted via powerline communication in the system 100 as well. In certain embodiments, the database 155 may serve as a central repository for any information associated with any of the devices and information associated with the system 100. Furthermore, the database 155 may include a processor and memory or be connected to a processor and memory to perform the various operations associated with the database 155. In certain embodiments, the database 155 may be connected to the servers 140, 150, 160, the first user device 102, the second user device 111, any devices in the system 100, any other device, any network, or any combination thereof.

The database 155 may also store information obtained from the system 100, store information associated with the first and second users 101, 110, store location information for the first and second user devices 102, 111 and/or first and second users 101, 110, store user profiles associated with the first and second users 101, 110, store device profiles associated with any device in the system 100 (e.g. the sensors used in the flexible mesh supporting the functionality of the system 100), store communications traversing the system 100, store user preferences, store demographic information for the first and second users 101, 110, store information associated with any device or signal in the system 100, store information relating to usage of applications accessed by the first and second user devices 102, 111, store any information obtained from any of the networks in the system 100, store historical data associated with the first and second users 101, 110, store device characteristics, store information relating to any devices associated with the first and second users 101, 110, or any combination thereof. The database 155 may store algorithms for analyzing sensor data obtained from the flexible sensors, algorithms conducting artificial intelligence and/or machine learning, any other algorithms for performing any other calculations and/or operations in the system 100, or any combination thereof. In certain embodiments, the database 155 may be configured to store any information generated and/or processed by the system 100, store any of the information disclosed for any of the operations and functions disclosed for the system 100 herewith, store any information traversing the system 100, or any combination thereof. Furthermore, the database 155 may be configured to process queries sent to it by any device in the system 100.

The system 100 may also include a software application, which may be configured to perform and/or support the operative functions of the system 100. In certain embodiments, the application may be a website, a mobile application, a software application, or a combination thereof, which may be made accessible to users utilizing one or more computing devices, such as first user device 102 and second user device 111. The application of the system 100 may be accessible via an internet connection established with a browser program executing on the first or second user devices 102, 111, a mobile application executing on the first or second user devices 102, 111, or through other suitable means. Additionally, the application may allow users and computing devices to create accounts with the application and sign-in to the created accounts with authenticating username and password log-in combinations. The application may include a custom graphical user interface that the first user 101 or second user 110 may interact with by utilizing a web browser executing on the first user device 102 or second user device 111. In certain embodiments, the software application may execute directly as an installed program on the first and/or second user devices 102, 111.

The software application may include multiple programs and/or functions that execute within the software application and/or are accessible by the software application. For example, the software application may include an application that generates web content, pages, and/or data that may be accessible to the first and/or second user devices 102, 111, the sensors, the database 155, the external network 165, any type of program, any device and/or component of the system 100, or any combination thereof. The application that generates web content and pages may be configured to generate a graphical user interface and/or other types of interfaces for the software application that is accessible and viewable by the first and second users 101, 110 when the software application is loaded and executed on the first and/or second computing devices 102, 111. The graphical user interface for the software application may display content associated with sensor data measured by the sensors of the flexible mesh device, any other type of information, or any combination thereof. The software application may also process and/or store measurements obtained from sensors of the flexible mesh circuit infrastructure. Additionally, the graphical user interface may display functionality provided by the software application that enables the first and/or second user 101, 110 and/or the first user device and/or second user device 111 to input parameters and requirements for the various processes conducted by the system 100.

The system 100 may also include an external network 165. The external network 165 of the system 100 may be configured to link each of the devices in the system 100 to one another. For example, the external network 165 may be utilized by the first user device 102, and/or the flexible sensor mesh infrastructure to connect with other devices within or outside communications network 135. Additionally, the external network 165 may be configured to transmit, generate, and receive any information and data traversing the system 100. In certain embodiments, the external network 165 may include any number of servers, databases, or other componentry, and may be controlled by a service provider. The external network 165 may also include and be connected to a cloud-computing network, a phone network, a wireless network, an Ethernet network, a satellite network, a broadband network, a cellular network, a private network, a cable network, the Internet, an internet protocol network, a content distribution network, any network, or any combination thereof. In certain embodiments, the external network 165 may be outside the system 100 and may be configured to perform various functionality provided by the system 100, such as if the system 100 is overloaded and/or needs additional processing resources. In certain embodiments, the external network 165 may be configured to perform some or all of the operations conducted by flexible mesh sensors.

As shown in FIG. 1, the system 100 may further include a Super Modular Monitoring System 2 that may be made up of at least one lattice 4, lattice intersections 6, a primary sensor 10, secondary sensors 12, and a monitoring system 14. The lattice 4 may be made of a flexible material that transmits both power and data and may be constructed of an array of repeating geometric patterns. The lattice 4 may include any number of primary and secondary sensors 10, 12, which may be arranged in a mesh configuration, as shown in FIG. 1 The size of lattice 4 may be determined by the limitations resulting from the relationship between the surface area to be monitored by the lattice 4 and the conductive, electrical, and physical properties of the lattice 4. Intersections 6 may be disposed at the junctions of lattice 4. The lattice 4 may be constructed and formed using the primary sensor 10 and at least one secondary sensor 12. A primary sensor 10 forms the layout and design on the lattice 4 and may be configured to integrate with one or more secondary sensors 12. For example, the one or more secondary sensors 12 may be disposed at any intersection 6 of the primary sensor 10 of the lattice 4. The entire lattice 4 may be electronically connected to a monitoring system 14 which accepts and processes both power and data transmissions from the lattice 4. A series of lattices 4 can be connected to cover great distances or span massive surfaces. A series of connected lattices 4 may comprise a super lattice 18. The Super Modular Monitoring System 2 may comprise a flexible mesh circuit infrastructure and may be utilized to monitor any object and/or substance upon which the Super Modular Monitoring System 2 is disposed.

Notably, as shown in FIG. 1, the system 100 may perform any of the operative functions disclosed herein by utilizing the processing capabilities of server 160, the storage capacity of the database 155, or any other component of the system 100 to perform the operative functions disclosed herein. The server 160 may include one or more processors 162 that may be configured to process any of the various functions of the system 100. The processors 162 may be software, hardware, or a combination of hardware and software. Additionally, the server 160 may also include a memory 161, which stores instructions that the processors 162 may execute to perform various operations of the system 100. For example, the server 160 may assist in processing loads handled by the various devices in the system 100, such as, but not limited to, operations conducted by the sensors of the flexible mesh circuit infrastructure, facilitating transmission of both power and data via the flexible mesh circuit infrastructure, determining if requirements have changed for the flexible mesh infrastructure, measuring biological and/or other data utilizing the sensors of the flexible mesh circuit infrastructure, and performing any other suitable operations conducted in the system 100 or otherwise. In one embodiment, multiple servers 160 may be utilized to process the functions of the system 100. The server 160 and other devices in the system 100, may utilize the database 155 for storing data about the devices in the system 100 or any other information that is associated with the system 100. In one embodiment, multiple databases 155 may be utilized to store data in the system 100.

In certain embodiments, the system 100 may also include a computing device 170. The computing device 170 may include one or more processors 172 that may be configured to process any of the various functions of the system 100. The processors 172 may be software, hardware, or a combination of hardware and software. Additionally, the computing device 170 may also include a memory 171, which stores instructions that the processors 172 may execute to perform various operations of the system 100. For example, the computing device 170 may assist in processing loads handled by the various devices in the system 100, such as, but not limited to, the flexible mesh and its sensors.

Although FIGS. 1-3 illustrates specific example configurations of the various components of the system 100, the system 100 may include any configuration of the components, which may include using a greater or lesser number of the components. For example, the system 100 is illustratively shown as including a first user device 102, a second user device 111, a communications network 135, a server 140, a server 150, a server 160, a database 155, an external network 165, and the Super Modular Monitoring System 2. However, the system 100 may include multiple first user devices 102, multiple second user devices 111, multiple databases 125, multiple communications networks 135, multiple servers 140, multiple servers 150, multiple servers 160, multiple databases 155, multiple external networks 165, multiple Super Modular Monitoring Systems 2, any number of components that are included in the Super Modular Monitoring System 2, and/or any number of any of the other components inside or outside the system 100. Similarly, the system 100 may include any number of data sources, applications, systems, and/or programs. Furthermore, in certain embodiments, substantial portions of the functionality and operations of the system 100 may be performed by other networks and systems that may be connected to system 100.

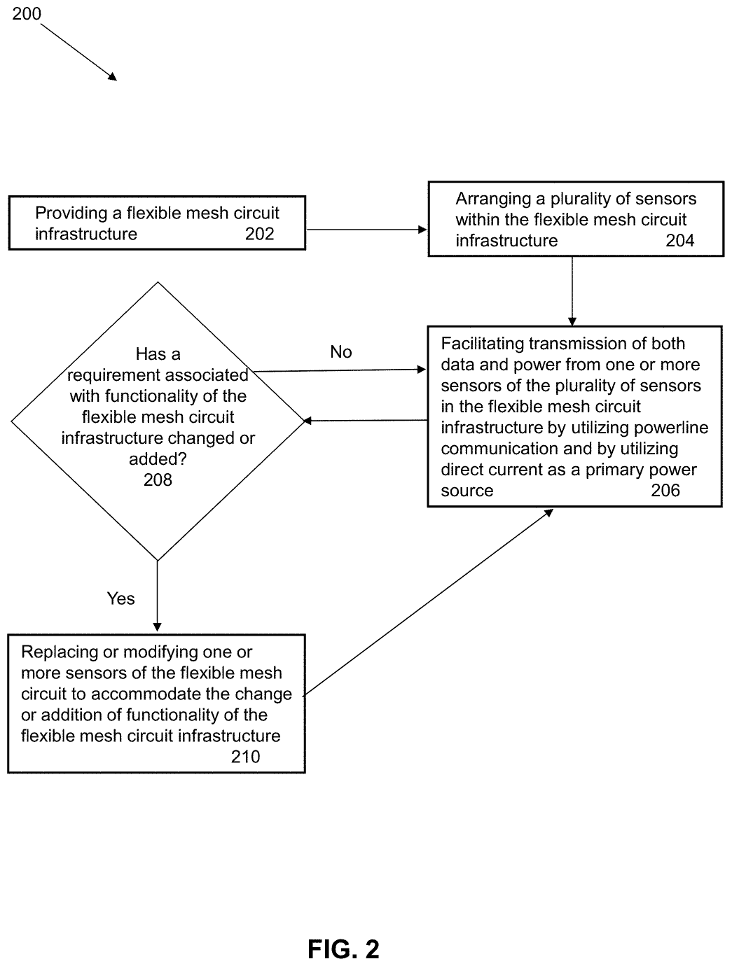

As shown in FIG. 2, an exemplary method 200 for providing powerline communication over flexible mesh for circuit design used in monitoring is schematically illustrated. The method 200 may include, at step 202, providing a flexible mesh circuit infrastructure. The flexible mesh circuit infrastructure may be comprised of flexible components, such as sensors, which may be arranged in a mesh configuration. In certain embodiments, the flexible mesh circuit infrastructure may be composed of and/or include one or more Super Modular Monitoring Systems 2, which may include any number of lattices 4, lattice intersections 6, primary sensors 10, secondary sensors 12, monitoring systems 14, and/or super lattices, such as is described in U.S. Provisional Patent Application 62/953,309, filed on Dec. 24, 2019, which is hereby incorporated by reference in its entirety. Notably, any of the functionality and/or features described in U.S. Provisional Patent Application 62/953,309 may be incorporated into the method 200. At step 204, the method 200 may include arranging and/or positioning one or more sensors within and/or on the flexible mesh circuit infrastructure. In certain embodiments, arranging and/or positioning may be performed and/or facilitated by utilizing the first user device 102, the second user device 111, the server 140, the server 150, the server 160, the communications network 135, the external network 165, the database 155, the computing device 170, any appropriate program, device, network, and/or process of the system 100, any user of the system 100, or a combination thereof.

At step 206, the method 200 may include facilitating transmission of both data and power from one or more of the sensors of the flexible mesh circuit infrastructure by utilizing powerline communication. In certain embodiments, the transmission of both data and power may be conducted by utilizing direct current as the primary power source for the flexible mesh circuit infrastructure. In certain embodiments, the transmission of both data and power may be conducted by utilizing direct current as the only power source for the flexible mesh circuit infrastructure. In certain embodiments, the transmission of both data and power may be conducted by utilizing any combination of power sources including alternating current power sources and/or other power sources. In certain embodiments, the transmission of both data and power may be performed and/or facilitated by utilizing any component(s) of the Super Modular Monitoring Systems 2, the first user device 102, the second user device 111, the server 140, the server 150, the server 160, the communications network 135, the external network 165, the database 155, the computing device 170, any appropriate program, device, network, and/or process of the system 100, any user of the system 100, or a combination thereof.

At step 208, the method 200 may include determining if a requirement associated with functionality of the flexible mesh circuit infrastructure has been changed, added, and/or removed. For example, if originally the flexible mesh circuit infrastructure was utilized to monitoring metrics such as body temperature, oxygen saturation levels, and heart rate, and now, the flexible mesh circuit infrastructure needs to also monitor blood pressure levels of a user, this may constitute a change in requirement for the flexible mesh circuit infrastructure. If, at step 208, it is determined that there is no change in requirements, the method 200 may revert to step 206 and continue to transmit both data and power in the flexible mesh circuit infrastructure using the current configuration. If, however, at step 208, it is determined that there is a change in a requirement for the flexible mesh circuit infrastructure, the method 200 may proceed to step 210, which may include replacing and/or modifying one or more sensors of the flexible mesh circuit to accommodate the change in requirement of the flexible mesh circuit configuration. Using the example above, one or more blood pressure sensors may be incorporated into the flexible mesh circuit infrastructure so that blood pressure measurements may be obtained and transmitted via the flexible mesh circuit infrastructure. To that end, the method 200 may proceed back to step 206 and may transmit both power and data using the new configuration for the flexible mesh circuit configuration. Notably, the method 200 may further incorporate any of the features and functionality described for the system 100 or as otherwise described herein.

The systems and methods disclosed herein may include additional functionality and features. For example, the operative functions of the system 100 and method may be configured to execute on a special-purpose processor specifically configured to carry out the operations provided by the system 100 and method. Notably, the operative features and functionality provided by the system 100 and method may increase the efficiency of computing devices that are being utilized to facilitate the functionality provided by the system 100 and method 200. For example, through the use of artificial intelligence and machine learning in conjunction with the system 100 and/or method 200, a reduced amount of computer operations would need to be performed by the devices in the system 100 using the processors and memories of the system 100 than in systems that are not capable of machine learning as described in this disclosure. As an illustration and in certain embodiments, the system 100 may learn over time that certain sensor measurements measured in the flexible mesh circuit infrastructure are associated with certain health conditions. For example, if the system 100 initially determines during a first occasion that measured biological data of a user is outside a threshold range of values associated with the safety of the user, the system 100 may determine the health condition associated with the user. Knowing this information, the system 100 may automatically determine the health condition for the user during a future second occasion if newly measured biological data matches or approximates the biological data from the first occasion. As a result, in such a scenario, the system 100 would not have to compare the newly measured biological data to the threshold range of values because the system 100 already has determined that the health condition would exist based on the measured data. In such a context, less processing power needs to be utilized because the processors and memories do not need perform analyses and operations that have already been learned by the system 100. As a result, there are substantial savings in the usage of computer resources by utilizing the software, functionality, and algorithms provided in the present disclosure.

Notably, in certain embodiments, various functions and features of the system 100 and methods may operate without human intervention and may be conducted entirely by computing devices, robots, and/or processes. For example, in certain embodiments, multiple computing devices may interact with devices of the system 100 to provide the functionality supported by the system 100. Additionally, in certain embodiments, the computing devices of the system 100 may operate continuously to reduce the possibility of errors being introduced into the system 100. In certain embodiments, the system 100 and methods may also provide effective computing resource management by utilizing the features and functions described in the present disclosure. For example, in certain embodiments, while facilitating transmission of both data and power using powerline communication, any selected device in the system 100 may transmit a signal to a system 100 component facilitating the transmission that only a specific quantity of computer processor resources (e.g. processor clock cycles, processor speed, processor cache, etc.) may be dedicated to transmitting data, processing any other operation conducted by the system 100, or any combination thereof. For example, the signal may indicate an amount of processor cycles of a processor that may be utilized to process the data generated in the flexible mesh circuit infrastructure, and/or specify a selected amount of processing power that may be dedicated to processing the data and/or any of the operations performed by the system 100. In certain embodiments, a signal indicating the specific amount of computer processor resources or computer memory resources to be utilized for performing an operation of the system 100 may be transmitted from the first and/or second user devices 102, 111 and/or the flexible mesh circuit infrastructure to the various components and devices of the system 100.

In certain embodiments, any device in the system 100 may transmit a signal to a memory device to cause the memory device to only dedicate a selected amount of memory resources to the various operations of the system 100. In certain embodiments, the system 100 and methods may also include transmitting signals to processors and memories to only perform the operative functions of the system 100 and methods at time periods when usage of processing resources and/or memory resources in the system 100 is at a selected, predetermined, and/or threshold value. In certain embodiments, the system 100 and methods may include transmitting signals to the memory devices utilized in the system 100, which indicate which specific portions (e.g. memory sectors, etc.) of the memory should be utilized to store any of the data utilized or generated by the system 100. Notably, the signals transmitted to the processors and memories may be utilized to optimize the usage of computing resources while executing the operations conducted by the system 100. As a result, such features provide substantial operational efficiencies and improvements over existing technologies.

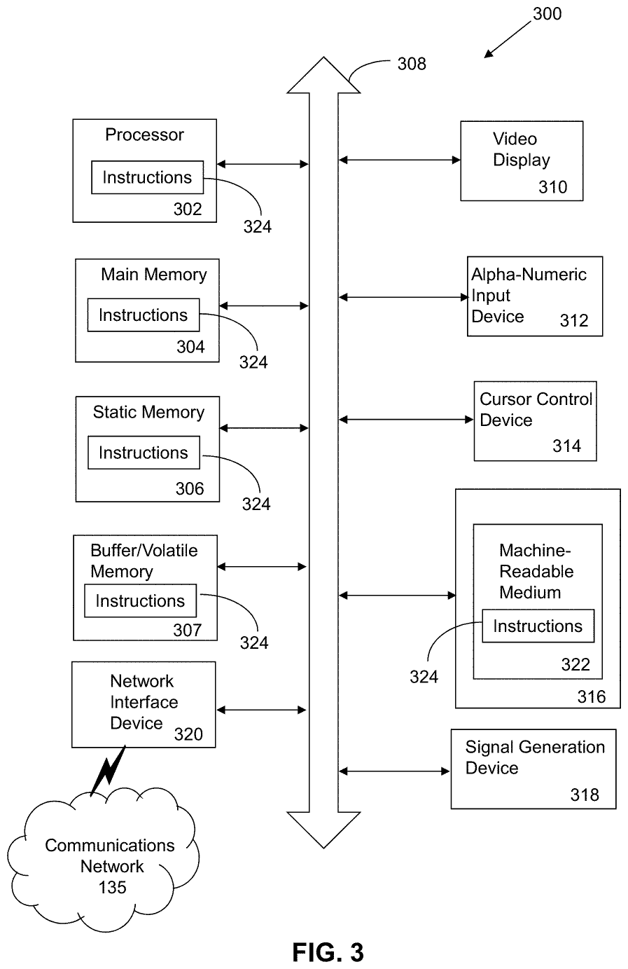

Referring now also to FIG. 3, at least a portion of the methodologies and techniques described with respect to the exemplary embodiments of the system 100 can incorporate a machine, such as, but not limited to, computer system 300, or other computing device within which a set of instructions, when executed, may cause the machine to perform any one or more of the methodologies or functions discussed above. The machine may be configured to facilitate various operations conducted by the system 100. For example, the machine may be configured to, but is not limited to, assist the system 100 by providing processing power to assist with processing loads experienced in the system 100, by providing storage capacity for storing instructions or data traversing the system 100, or by assisting with any other operations conducted by or within the system 100.

In some embodiments, the machine may operate as a standalone device. In some embodiments, the machine may be connected (e.g., using communications network 135, another network, or a combination thereof) to and assist with operations performed by other machines, programs, functions, and systems, such as, but not limited to, the first user device 102, the second user device 111, the server 140, the server 150, the database 155, the server 160, any component of the Super Modular Monitoring System 2, the external network 165, the communications network 135, any device, system, and/or program in FIGS. 1-3, or any combination thereof. The machine may be connected with any component in the system 100. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 300 may include a processor 302 (e.g., a central processing unit (CPU), a graphics processing unit (GPU, or both), a main memory 304, and a static memory 306, and/or a buffer and/or volatile memory 307, which communicate with each other via a bus 308. The computer system 300 may further include a video display unit 310, which may be, but is not limited to, a liquid crystal display (LCD), a flat panel, a solid state display, a touch screen monitor, or a cathode ray tube (CRT). The computer system 300 may include an input device 312, such as, but not limited to, a keyboard, a cursor control device 314, such as, but not limited to, a mouse, a disk drive unit 316, a signal generation device 318, such as, but not limited to, a speaker or remote control, and a network interface device 320.

The disk drive unit 316 may include a machine-readable medium 322 on which is stored one or more sets of instructions 324, such as, but not limited to, software embodying any one or more of the methodologies or functions described herein, including those methods illustrated above. The instructions 324 may also reside, completely or at least partially, within the main memory 304, the static memory 306, or within the processor 302, or a combination thereof, during execution thereof by the computer system 300. The main memory 304 and the processor 302 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 322 containing instructions 324 so that a device connected to the communications network 135, the external network 165, another network, or a combination thereof, can send or receive voice, video or data, and communicate over the communications network 135, the external network 165, another network, or a combination thereof, using the instructions. The instructions 324 may further be transmitted or received over the communications network 135, the external network 165, another network, or a combination thereof, via the network interface device 320.

While the machine-readable medium 322 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this invention, but that the invention will include all embodiments and arrangements falling within the scope of the appended claims.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

1. A system, comprising:
a flexible mesh circuit infrastructure, the flexible mesh circuit infrastructure comprising:
a plurality of sensors arranged in the flexible mesh circuit infrastructure, wherein each sensor of the plurality of sensors is configured to transmit and receive data and power in the flexible mesh circuit infrastructure using powerline communication and by using direct current as a primary power source.

2. The system of claim 1, wherein the data includes information collected by the plurality of sensors relevant to a change in a circuit of the flexible mesh circuit infrastructure.

3. The system of claim 1, wherein the flexible mesh circuit infrastructure supports distributed powerline communication over the direct current.

4. The system of claim 1, wherein the system operates without requiring a hub for managing compression, transmission, or receipt of the data in the flexible mesh circuit infrastructure.

5. The system of claim 1, wherein the system is configured to support digital data burst over electrical infrastructure.

6. The system of claim 1, wherein the system is configured to support coded, multiplexed analog to digital/digital to analog transmission.

7. The system of claim 1, wherein the system is configured to account for a change in an electrical property associated with a distortion of a conductive elastomer utilized in the flexible mesh circuit infrastructure.

8. The system of claim 1, wherein each sensor of the plurality of sensors is configured to transmit and receive the data and the power in a flexible geometric system.

9. The system of claim 1, wherein the system is configured to facilitate a mapping of freedom and constraint topology.

10. A method, comprising:
providing a flexible mesh circuit infrastructure;
arranging a plurality of sensors within the flexible mesh circuit infrastructure; and
facilitating transmission and receipt of data and power from each sensor of the plurality of sensors in the flexible mesh circuit infrastructure by utilizing powerline communication and by utilizing direct current as a primary power source.

11. The method of claim 10, wherein receipt of the data comprises receiving information relevant to a change in a circuit of the flexible mesh circuit infrastructure.

12. The method of claim 10, further comprising facilitating digital data burst.

13. The method of claim 10, further comprising facilitating the transmission and the receipt of the data and power without requiring a hub.

14. The method of claim 10, further comprising determining a change in an electrical property associated with a conductive elastomer of the flexible mesh circuit infrastructure.

15. The method of claim 10, further comprising performing a mapping of freedom and constraint topology.

16. The method of claim 10, further comprising enabling coded, multiplexed analog to digital/digital to analog transmission via the flexible mesh circuit infrastructure.

17. The method of claim 10, further comprising managing compression of the data via the flexible mesh circuit infrastructure.

18. The method of claim 10, further comprising enabling distributed powerline communication over the direct current.

19. The method of claim 10, further comprising replacing a sensor of the plurality of sensors based on a change in a requirement for the flexible mesh circuit infrastructure.

20. A flexible mesh circuit infrastructure, comprising:
a plurality of sensors, wherein each sensor of the plurality of sensors is configured to transmit and receive data and power using powerline communication and by using direct current as a primary power source.
